(19) United States
(12) Patent Application Publication
(10) Pub. No.: US 2025/0002937 A1
(43) Pub. Date: Jan. 2, 2025

(54) EBOLA PSEUDOTYPED VECTORS AND METHODS OF USE THEREOF

(71) Applicant: Interius BioTherapeutics, Inc., Philadelphia, PA (US)

(72) Inventors: Philip R. Johnson, Sarasota, FL (US); Richard W. Peluso, Lansdale, PA (US); Ronnie M. Russell, Philadelphia, PA (US); Bruce C. Schnepp, Havertown, PA (US)

(21) Appl. No.: 18/701,161

(22) PCT Filed: Oct. 14, 2022
(86) PCT No.: PCT/US2022/078092

§ 371 Date: Apr. 12, 2024

Related U.S. Application Data
(60) Provisional application No. 63/256,447, filed on Oct. 15, 2021.

(51) Int. Cl. C12N 15/86; A61K 35/17; A61K 48/00; C07K 14/005; C12N 5/0783

(52) U.S. Cl. CPC C12N 15/86; A61K 35/17; A61K 48/005; C07K 14/005; C12N 5/0636; C12N 2740/15042; C12N 2740/15043; C12N 2740/15045; C12N 2760/14122

(57) ABSTRACT

The present embodiments provide methods and compositions for pseudotyping viral vectors. Also provided are methods and compositions for creating targeting moiety fused viral glycoproteins. Also provided herein are methods of treating a disease in a subject in need thereof using the compositions provided for herein. Also provided are methods of delivering a molecule of interest to a target cell using the compositions provided for herein.

### BACKGROUND

Recombinant virus-based vectors have been used as a mode of gene delivery to host cells. Through induction of a desired adaptive immune response, the expressed gene product provides therapeutic benefit. There are a number of challenges however to achieving a safe and effective system. Some of these challenges include designing a vector that targets a desired set of host cells, providing a suitable delivery system, expressing a desired antigen to elicit an effective immune response and consistently manufacturing a sufficiently high tittered pharmaceutical composition of the recombinant viral vector so that it can be utilized broadly across a designated human subject population. The present disclosure fulfills these needs as well as others.

### SUMMARY

Disclosed herein are engineered viral particles, methods, compositions, and polypeptides that allow delivery of molecules to target cells. Embodiments disclosed herein are incorporated by reference into this section.

In some embodiments, engineered viral particles comprising an engineered envelope comprising a recombinant Ebola virus glycoprotein, or a variant thereof, a gag-pol protein, and an engineered targeting moiety for binding to a target cell; and a nucleic acid encoding a polypeptide of interest, are provided.

In some embodiments, engineered viral particles comprising an engineered envelope comprising an Ebola virus glycoprotein fused or linked to a targeting moiety, and a gag-pol protein, wherein the Ebola virus glycoprotein fused or linked to a targeting moiety has the amino acid sequence as set forth in SEQ ID NO: 5-9; and a nucleic acid encoding a polypeptide of interest, are provided.

In some embodiments, polypeptide molecules comprising a targeting moiety fused or linked to an Ebola virus glycoprotein in place of the glycan cap, and/or the MLD of the Ebola virus glycoprotein, are provided.

In some embodiments, polypeptide molecules comprising an EBOV GP, or a variant thereof, fused to a targeting moiety via a linker, such as a peptide linker, and having a sequence as set forth in SEQ ID NO: 5-9, are provided.

In some embodiments, methods of in vivo delivery of a molecule of interest to a target cell, the method comprising administering an engineered viral particle to a subject in need of delivery, wherein the engineered viral particle comprises an engineered envelope comprising a recombinant Ebola virus glycoprotein, or a variant thereof, a gag-pol protein, and an engineered targeting moiety for binding to the target cell; a nucleic acid encoding the molecule of interest; and wherein the administration of the engineered viral particle delivers the nucleic acid molecule encoding the molecule of interest to the cell, are provided.

In some embodiments, methods of in vivo delivery of a molecule of interest to a target cell, the method comprising administering an engineered viral particle to a subject in need of delivery, wherein the engineered viral particle comprises an engineered envelope comprising an Ebola virus glycoprotein fused or linked to a targeting moiety, and a gag-pol protein, wherein the Ebola virus glycoprotein fused or linked to the targeting moiety has the amino acid sequence as set forth in SEQ ID NO: 5-9; a nucleic acid encoding a molecule of interest; and wherein the administration of the engineered viral particle delivers the nucleic acid molecule encoding the molecule of interest to the cell, are provided.

In some embodiments, methods of delivering a peptide of interest to a target cell, such as an antigen presenting cell (“APC”), the method comprising contacting an engineered viral particle as provided herein for binding to a target cell with an APC, thereby delivering the peptide of interest to the APC, are provided.

In some embodiments, methods of treating a disease in a subject, the method comprising administering to the subject the engineered viral particle as provided herein, wherein the engineered viral particle expresses the polypeptide of interest in a cell, are provided.

### BRIEF DESCRIPTION OF THE DRAWINGS

FIG. 1A illustrates the recognition site of mAb KZ52. mAb KZ52 recognizes a conformational pre-fusion epitope spanning GP1/GP2.

FIG. 1B illustrates HEK293T cells transfected to produce eGFP-carrying lentiviruses pseudotyped with various Ebola GP constructs, stained and analyzed at the time of virus harvest for the expression of the eGFP transgene (x-axis) and Ebola GP (y-axis).

FIG. 2 shows CD8+ SupT1 cells transduced with lentivirus pseudotyped with various Ebola GP constructs and analyzed for GFP and CD8 expression four days post transduction. FIG. 2A illustrates the flowcytometry results. FIG. 2B illustrates the quantification of FIG. 2A.

FIG. 3 shows CD8-293T cells transduced with lentivirus pseudotyped with various Ebola GP constructs and analyzed for GFP and CD8 expression four days post transduction. FIG. 3A illustrates the flowcytometry results. FIG. 2B illustrates the quantification of FIG. 3A.

### DETAILED DESCRIPTION

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

It must also be noted that as used herein, the singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50% means in the range of 45%-55%. The present disclosure modifies certain terms or values with the term “about,” however, the disclosure should also be understood to disclose the exact value as well and is simply not written out for convenience. For example, the phrase “about 9 to about 25” also discloses “9 to 25.” Additionally, a range, such the phrase “from X to Y” where X and Y are any integer includes the endpoints. For example, the phrase “from 1 to 5” means 1, 2, 3, 4, or 5.

“Activation,” as used herein in reference to a T cell, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

“Administering” when used in conjunction with a therapeutic means to administer a therapeutic directly into or onto a target tissue or to administer a therapeutic to a patient. Non-limiting examples of methods of administration that can be used to administer nucleic acid molecules, include, but are not limited to, transfection, electroporation, injection, sonication, or by any method in combination with other known techniques. Such combination techniques include heating and radiation. In some embodiments, the nucleic acid molecule is delivered to a muscle cell. This can be done, for example, by electroporation or other suitable technique. Electroporation of the nucleic acid molecule to the muscle or other tissue type can be done, for example, using an electroporation device.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. The term “antigen” can also refer to a molecule that an antibody or antibody-like molecule can bind to or is recognized by the antibody or antibody-like molecule.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material, such as a cell, derived from a subject to which it is later to be re-introduced into the same subject.

As used herein, the term “allogeneic” is meant to refer to material, such as a cell, derived from one subject that is later introduced into a different subject.

The term “animal” as used herein includes, but is not limited to, humans and non-human vertebrates such as wild, domestic and farm animals.

A “co-stimulatory molecule” refers to the cognate binding partner on a T cell that specifically binds with a co-stimulatory ligand, thereby mediating a co-stimulatory response by the T cell, such as, but not limited to, proliferation, activation, differentiation, and the like.

The term “cloning” is used in reference to the ligating process of a nucleic acid molecule into another nucleic acid molecule, such as a plasmid. The cloned molecule can then be transferred into a host cell or subject for duplication, amplification, or administration.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

“Identity” as used herein refers to the subunit sequence identity between two polymeric molecules, such as between two amino acid molecules, such as, between two polypeptide molecules, or two nucleic acid molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. Percent identity can be determined using BLASTP or other similar algorithms, using default parameters. Examples of BLASTP can be found at the National Center for Biotechnology Information website.

As used herein, when an amino acid or nucleotide position is made in reference to a reference sequence or is said “compared to” another polypeptide or nucleotide sequence, it is based on an alignment that can be done using BLASTP or BLASTN or CLUSTAL with default settings or if multiple sequences are being aligned it can be performed with a program based on CLUSTAL.

The term “immune response” as used herein is defined as a cellular response to an antigen that occurs when lymphocytes identify antigenic molecules as foreign and induce the formation of antibodies and/or activate lymphocytes to remove the antigen. In some embodiments, the immune response can be against a tumor cell expressing the antigen. In some embodiments, the immune response is facilitated by a T cell expressing a chimeric antigen receptor, such as those, but not limited to, those provided herein.

The term “immunosuppressive” is used herein to refer to reducing overall immune response.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Lentiviruses can deliver a significant amount of genetic information into the DNA of the host cell, so they are one of the most efficient methods of a gene delivery vector. HIV, SIV, and FIV are all examples of lentiviruses. Vectors derived from lentiviruses offer the ability to achieve gene transfer in vivo.

By the term “modified” as used herein, is meant a changed state or structure of a molecule or cell as provided herein. Molecules may be modified in many ways, including chemically, structurally, and functionally. Cells may be modified through the introduction of nucleic acids or the expression of heterologous proteins.

As used herein, the following abbreviations for the commonly occurring nucleic acid bases are used: “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing envelope glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a HIV lentiviral vector can have the HIV envelope glycoprotein be replaced with the VSV envelope glycoprotein. This is just one non-limiting example and other envelop glycoproteins can be used, such as the glycoprotein of the Ebola virus. Therefore, in some embodiments, the viral particle is encoded by a lentivirus that encodes the Ebola virus glycoprotein.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” includes living organisms, including those in which an immune response can be elicited (e.g., mammals). A “subject” or “patient,” as used therein, may be a human or non-human mammal. Non-human mammals include, for example, livestock and pets, such as ovine, bovine, porcine, canine, non-human primates, feline and murine mammals. In some embodiments, the subject is human.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

To “treat” a disease as the term is used herein, means to reduce the frequency or severity of at least one sign or symptom of a disease or disorder experienced by a subject.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution. In some embodiments, the variant comprises a deletion. In some embodiments, the variant comprises an insertion. In some embodiments, the variant comprises a substitution, insertion, or deletion, or any combination thereof.

As used herein, when a polypeptide, nucleic acid molecule, or amino acid sequence is “as compared to a SEQ ID NO:”, it is to be understood that is the equivalent of stating that the polypeptide or nucleotide sequence is “as compared to polypeptide/nucleic acid molecule comprising the amino acid/nucleic acid sequence of” the referenced sequence identifier.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, retroviral vectors, lentiviral vectors, and the like.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

### Viruses

In some embodiments, the virus particle is a lentiviral particle. Lentiviral particles are derived from lentiviruses, which are retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral particles have been generated by multiply attenuating the HIV virulence genes, for example, the genes env, vif, vpr, vpu and nef are deleted making the vector biologically safe. Lentiviral particles are capable of infecting non-dividing cells and can be used for both in vivo and ex vivo gene transfer and expression, e.g., of a nucleic acid encoding a CAR (see, e.g., U.S. Pat. No. 5,994,136).

Retrovirus expression vectors are capable of integrating into the host genome, delivering a large amount of foreign genetic material, infecting a broad spectrum of species and cell types and being packaged in special cell lines. The retroviral particle is constructed by inserting a nucleic acid (e.g., a nucleic acid encoding a CAR) into the viral genome at certain locations to produce a virus that is replication defective. Though the retroviral particles are able to infect a broad variety of cell types, integration and stable expression of the CAR requires the division of host cells.

### Engineered Viral Particles

The present invention provides, among other things, compositions that can be used, for example, to selectively transduce a cell expressing specific cell surface markers in vivo using viral particles. By utilizing viral particles to transduce the same cell type, the viral particles can increase specificity, or improve safety by limiting expression of the entire molecule of interest to the specific cell type.

The present invention also provides, among other things, compositions that can be used, for example, to selectively transduce a cell expressing specific cell surface markers in vivo using a plurality of viral particles. By utilizing a plurality of viral particles to transduce the same cell type, the viral particles can increase specificity, or improve safety by limiting expression of the entire molecule of interest to the specific cell type.

In some embodiments, the viral particle encodes a polypeptide of interest, which can also be referred to as a molecule of interest. In some embodiments, the examples of polypeptides of interest include, but are not limited to, chimeric antigen receptors (CAR), vaccines, or gene therapies.

In some embodiments, a composition comprising an engineered viral particle comprising an engineered envelope harboring a glycoprotein, or a variant thereof, a chimeric gag protein, and an engineered targeting moiety for binding to a target cell; and a nucleic acid encoding a polypeptide of interest is provided.

In some embodiments, an engineered viral particle comprising an engineered envelope comprising an Ebola virus glycoprotein, or a variant thereof, a gag-pol protein, and an engineered targeting moiety for binding to a target cell; and a nucleic acid encoding a polypeptide of interest is provided. In some embodiments, the targeting moiety is fused to the Ebola virus glycoprotein. In some embodiments, the viral particle is a lentivirus.

In some embodiments, the glycoprotein is an Ebola virus (EBOV) glycoprotein (GP). Without wishing to be bound to a particular theory, EBOV binding to the cell surface is independent from fusion. Binding occurs at the cell surface by receptors that bind EBOV particles through interactions with either virion-associated phosphatidylserine or viral glycoprotein glycans. EBOV is internalized through ruffling of the plasma membrane and macropinocytosis. During trafficking through endosomes, the EBOV glycoprotein is cleaved by proteases that remove the mucin-like domain (MLD) and glycan cap, exposing the receptor binding domain (RBD). The RBD interacts with endosomal receptor Niemann-Pick C1 (NPC1) in the late endosome/lysosome and triggers the release of the fusion loop, allowing for its insertion into the target membrane, which leads to fusion and release of the viral nucleoprotein into the cytoplasm. (Moller-Tank, S., & Maury, W. Ebola virus entry: a curious and complex series of events. PLoS Pathogens, 11(4), e1004731 (2015)). The function of the EBOV glycoprotein is maintained when the MLD is deleted and replaced with an exogenous protein. (Ao, Z., et al. Development and Evaluation of an Ebola Virus Glycoprotein Mucin-Like Domain Replacement System as a New Dendritic Cell-Targeting Vaccine Approach against HIV-1. Journal of Virology, 95(15), e0236820 (2021)).

In some embodiments, the Ebola virus glycoprotein comprises a signal peptide, a receptor binding domain, a cathepsin cleavage loop, a glycan cap, a mucin-like domain (MLD), a furin cleavage site, a fusion loop, a heptad repeat 1 (HR1), a heptad repeat 2 (HR2), a transmembrane domain, and a cytoplasmic tail. (Lee J. E., et al., Structure of the Ebola virus glycoprotein bound to an antibody from a human survivor. Nature Vol 454, 177-183 (2008)) In some embodiments, the Ebola virus glycoprotein comprises an amino acid sequence as set forth in SEQ ID NO: 1.

(SEQ ID NO: 1)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVSNGAKNISGQSPAR
TSSDPGTNTTTEDHKIMASENSSAMVQVHSQGREAAVSHLTTLATISTSPQSLTTKPGPDNSTHN
TPVYKLDISEATQVEQHHRRTDNDSTASDTPSATTAAGPPKAENTNTSKSTDFLDPATTTSPQNH
SETAGNNNTHHQDTGEESASSGKLGLITNTIAGVAGLITGGRRTRREAIVNAQPKCNPNLHYWTT
QDEGAAIGLAWIPYFGPAAEGIYIEGLMHNQDGLICGLRQLANETTQALQLFLRATTELRTFSIL
NRKAIDFLLQRWGGTCHILGPDCCIEPHDWTKNITDKIDQIIHDFVDKTLPDQGDNDNWWTGWRQ
WIPAGIGVTGVIIAVIALFCICKFVF

In some embodiments, the Ebola virus glycoprotein comprises a sequence comprising a deletion, an insertion, a mutation, or any combination thereof as compared to SEQ ID NO: 1. In some embodiments, the deletion comprises an amino acid deletion of a glycan cap sequence, a mucin-like domain (MLD) sequence, or any combination thereof. In some embodiments, the deletion comprises an amino acid deletion from or between positions 213-306, 305-484, 213-484, 213-497, and 232-497, as compared to SEQ ID NO: 1. In some embodiments, the deletion comprises a deletion of the glycan cap amino acid sequence. In some embodiments, the deletion of the glycan cap sequence comprises a deletion of the amino acid residues from or between positions 213 and 306 as compared to SEQ ID NO: 1. In some embodiments, the deletion comprises a deletion of a portion of the glycan cap amino acid sequence. In some embodiments, the deletion of a portion of the glycan cap amino acid sequence comprises deletion of amino acid residues from or between positions 232 and 306 as compared to SEQ ID NO: 1. In some embodiments, the deletion comprises a deletion of the MLD amino acid sequence. In some embodiments, the deletion of the MLD comprises a deletion of amino acid residues from or between positions 305 and 497 as compared to SEQ ID NO: 1. In some embodiments, the deletion comprises a deletion of a portion of the MLD amino acid sequence. In some embodiments, the deletion of a portion of the MLD amino acid sequence comprises a deletion of amino acid residues from or between positions 305 and 484 as compared to SEQ ID NO: 1. In some embodiments, the deletion comprises a deletion of the glycan cap amino acid sequence and the MLD amino acid sequence. In some embodiments, the deletion of the glycan cap and the MLD comprises a deletion of amino acid residues from or between positions 213 and 484 as compared to SEQ ID NO: 1. In some embodiments, the deletion of the glycan cap and the MLD comprises a deletion of amino acid residues from or between positions 232 and 497 as compared to SEQ ID NO: 1. In some embodiments, the deletion comprises an amino acid deletion from or between positions 213-306, 305-484, 213-484, 213-497, and 232-497, as compared to SEQ ID NO: 1.

In some embodiments, the insertion comprises an insertion in place of the glycan cap amino acid sequence, a mucin-like domain (MLD) amino acid sequence, or any combination thereof. In some embodiments, the insertion comprises an insertion in place of the glycan cap amino acid sequence. In some embodiments, the insertion in place of the glycan cap amino acid sequence comprises an insertion from or between amino acid residues at positions 213 and 306 as compared to SEQ ID NO: 1. In some embodiments, the insertion in place of the glycan cap amino acid sequence comprises an insertion from or between amino acid residues at positions 232 and 306 as compared to SEQ ID NO: 1. In some embodiments, the insertion comprises an insertion in place of the MLD amino acid sequence. In some embodiments, the insertion in place of the MLD amino acid sequence comprises an insertion from or between amino acid residues at positions 305 and 497 as compared to SEQ ID NO: 1. In some embodiments, the insertion in place of the MLD amino acid sequence comprises an insertion from or between amino acid residues at positions 305 and 484 as compared to SEQ ID NO: 1. In some embodiments, the insertion comprises an insertion in place of the glycan cap amino acid sequence and the MLD amino acid sequence. In some embodiments, the insertion in place of the glycan cap amino acid sequence and the MLD amino acid sequence comprises an insertion from or between amino acid residues at positions 213 and 484 as compared to SEQ ID NO: 1. In some embodiments, the insertion in place of the glycan cap amino acid sequence and the MLD amino acid sequence comprises an insertion from or between amino acid residues at positions 213 and 497 as compared to SEQ ID NO: 1. In some embodiments, the insertion in place of the glycan cap amino acid sequence and the MLD amino acid sequence comprises an insertion from or between amino acid residues at positions 232 and 497 as compared to SEQ ID NO: 1.

In some embodiments, the mutation comprises an insertion of the targeting moiety amino acid sequence in place of the glycan cap amino acid sequence, a mucin-like domain (MLD) amino acid sequence, or any combination thereof. In some embodiments, the mutation comprises an insertion of the targeting moiety amino acid sequence in place of the glycan cap amino acid sequence. In some embodiments, the mutation comprises an insertion of a targeting moiety amino acid sequence in place of the amino acid sequence from or between positions 213-306 as compared to SEQ ID NO: 1; an insertion of a targeting moiety amino acid sequence in place of the amino acid sequence from or between positions 305-484 as compared to SEQ ID NO: 1; an insertion of a targeting moiety amino acid sequence in place of the amino acid sequence from or between positions 213-484 as compared to SEQ ID NO: 1; an insertion of a targeting moiety amino acid sequence in place of the amino acid sequence from or between positions 213-497 as compared to SEQ ID NO: 1; or an insertion of a targeting moiety amino acid sequence in place of the amino acid sequence from or between positions 232-497 as compared to SEQ ID NO: 1. In some embodiments, the insertion of the targeting moiety amino acid sequence in place of the glycan cap amino acid sequence comprises an insertion from or between amino acid residues at positions 213 and 306 as compared to SEQ ID NO: 1. In some embodiments, the insertion of the targeting moiety amino acid sequence in place of the glycan cap amino acid sequence comprises an insertion from or between amino acid residues at positions 232 and 306 as compared to SEQ ID NO: 1. In some embodiments, the mutation comprises an insertion of the targeting moiety amino acid sequence in place of the MLD amino acid sequence. In some embodiments, the insertion of the targeting moiety amino acid sequence in place of the MLD amino acid sequence comprises an insertion from or between amino acid residues at positions 305 and 497 as compared to SEQ ID NO: 1. In some embodiments, the insertion of the targeting moiety amino acid sequence in place of the MLD amino acid sequence comprises an insertion from or between amino acid residues at positions 305 and 484 as compared to SEQ ID NO: 1. In some embodiments, the mutation comprises an insertion of the targeting moiety amino acid sequence in place of the glycan cap amino acid sequence and the MLD amino acid sequence. In some embodiments, the insertion of the targeting moiety amino acid sequence in place of the glycan cap amino acid sequence and the MLD amino acid sequence comprises an insertion from or between amino acid residues at positions 213 and 484 as compared to SEQ ID NO: 1. In some embodiments, the insertion of the targeting moiety amino acid sequence in place of the glycan cap amino acid sequence and the MLD amino acid sequence comprises an insertion from or between amino acid residues at positions 213 and 497 as compared to SEQ ID NO: 1. In some embodiments, the insertion of the targeting moiety amino acid sequence in place of the glycan cap amino acid sequence and the MLD amino acid sequence comprises an insertion from or between amino acid residues at positions 232 and 497 as compared to SEQ ID NO: 1. In some embodiments, the mutation comprises an amino acid insertion at any position from or between positions 213-306, 305-484, 213-484, 213-497, or 232-497, as compared to SEQ ID NO: 1.

In some embodiments, examples of the polypeptide of interest include, but are not limited to, a CAR, a vaccine, or a gene therapy. In some embodiments, the CAR comprises an extracellular (antigen binding) domain, a transmembrane domain, and an intracellular signaling domain.

In some embodiments, the extracellular/antigen binding domain is a domain that binds a tumor antigen (e.g. anti-CD19 scFv, anti-CD19 antibody, anti-CD33 scFv, and the like). In some embodiments, polypeptide of interest comprises the transmembrane domain and the CD3 zeta domain. In some embodiments, the CAR comprises an intracellular 4-1BB domain.

In some embodiments, the polypeptide of interest is a hemoglobin beta chain.

In some embodiments, a polypeptide molecule comprising a targeting moiety fused to an Ebola virus glycoprotein in place of the glycan cap, and/or the MLD of the Ebola virus glycoprotein is provided. In some embodiments, the targeting moiety is fused to Ebola virus glycoprotein via a linker. In some embodiments, the linker is a peptide linker. In some embodiments, the linker is a glycine/serine linker. In some embodiments, the targeting moiety is fused to the Ebola virus glycoprotein at a position from or between positions 213-306, 305-484, 213-484, 213-497, and 232-497, as compared to SEQ ID NO: 1. In some embodiments, the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the targeting moiety is a CD7 binding moiety. In some embodiments, the CD7 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the CD7 binding moiety is an anti-CD7 DARPin. In some embodiments, the targeting moiety is a CD8 binding moiety. In some embodiments, the CD8 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the CD8 binding moiety is an anti-CD8 DARPin. In some embodiments, the anti-CD8 DARPin comprises a sequence as set forth in SEQ ID NO: 2. In some embodiments, a polypeptide molecule comprising an EBOV GP, or a variant thereof, fused to a targeting moiety via a linker, and having a sequence as set forth in SEQ ID NO: 5-9 is provided. In some embodiments, a polypeptide molecule comprising an EBOV GP, or a variant thereof, fused to a targeting moiety via a linker, and having a sequence as set forth in SEQ ID NO: 5 is provided. In some embodiments, a polypeptide molecule comprising an EBOV GP, or a variant thereof, fused to a targeting moiety via a linker, and having a sequence as set forth in SEQ ID NO: 6 is provided. In some embodiments, a polypeptide molecule comprising an EBOV GP, or a variant thereof, fused to a targeting moiety via a linker, and having a sequence as set forth in SEQ ID NO: 7 is provided. In some embodiments, a polypeptide molecule comprising an EBOV GP, or a variant thereof, fused to a targeting moiety via a linker, and having a sequence as set forth in SEQ ID NO: 8 is provided. In some embodiments, a polypeptide molecule comprising an EBOV GP, or a variant thereof, fused to a targeting moiety via a linker, and having a sequence as set forth in SEQ ID NO: 9 is provided.

### Pseudotyped Viral Particles

To confer specificity of the viral particles to target cells, viral particles can be pseudotyped. Capsid proteins and envelope glycoproteins are implicated in virus attachment and interactions with cellular receptors, determining cell tropism. Manipulation of these viral surface proteins therefore may improve the transduction capacity of these vectors, expanding or restricting their tropism. Furthermore, experiments with vector pseudotyping demonstrated that pseudotyped vectors could achieve higher transduction titers and increase transduction efficacy.

In some embodiments, a virus particle comprising a targeting moiety that binds to a target on a cell and a nucleic acid molecule that encodes a polypeptide of interest is provided.

The targeting moiety can be any type of targeting moiety, including but not limited to, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof, a Centryn, Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the target is cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

In some embodiments, the targeting moiety is a protein that binds to a target, an antibody, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety is Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the targeting moiety binds to CD7. In some embodiments, the targeting moiety that binds to CD7 is an anti-CD7 antibody. In some embodiments, the anti-CD7 antibody is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety that binds to CD7 is an anti-CD7 DARPin. In some embodiments, the targeting moiety binds to CD8. In some embodiments, the targeting moiety that binds to CD8 is an anti-CD8 antibody. In some embodiments, the anti-CD8 antibody is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety that binds to CD8 is an anti-CD8 DARPin. In some embodiments, the anti-CD8 DARPin has the sequence as set forth in SEQ ID NO: 2 (PMID: 32160795 DOI: 10.1089/hum.2019.248).

(SEQ ID NO: 2)
DLGKKLLEASRAGQDDEVRILMANGADVNAQDRYGTTPLHLAAWHGHLEIVEVLLKHGADVNAND
VKGNTPLHLAANVGHLEIVEVLLKYGADVNAADNWGHTPLHLAAFWGHLEIVEVLLKYGADVNAQ
DKFGKTPFDLAIDNGNEDIAEVLQKAA

In some embodiments, the engineered virus particle is a pseudotyped virus particle. In some embodiments, the engineered virus particle is a pseudotyped lentiviral virus particle. In some embodiments, the pseudotyped lentiviral virus particle is pseudotyped with an ebolavirus glycoprotein, such as an Ebola virus glycoprotein. In some embodiments, the Ebola virus glycoprotein is mutated. In some embodiments, the engineered virus particle is pseudotyped with an Ebola virus glycoprotein. In some embodiments, the Ebola virus glycoprotein mutations comprise, but are not limited to, the mutations provided herein. Examples of viral particles pseudotyped with the Ebola virus glycoprotein, or a variant thereof, can be found in U.S. Pat. No. 7,981,656, and PCT Publication No. WO2019113688, each of which is hereby incorporated by reference in its entirety.

In some embodiments, the pseudotyped viral particle comprises a glycoprotein of an ebolavirus, wherein the glycan cap and/or MLD portions are truncated or deleted and wherein the truncated or deleted portion comprises an insertion of a targeting moiety. In some embodiments, the ebolavirus is an Ebola virus. In some embodiments, the targeting moiety comprises a linker sequence. In some embodiments, the linker is a peptide linker. In some embodiments, the linker sequence is a glycine/serine linker. In some embodiments, the linker has the sequence of SAGGGGSGGGGSGGGGSA (SEQ ID NO: 3) or TGGGGGSGGGGSGGGGSSA (SEQ ID NO: 4). In some embodiments, the targeting moiety is an antibody, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety is directed against the cell surface proteins as provided herein, including but not limited to, CD8. In some embodiments, the targeting moiety is directed against the cell surface proteins as provided herein, including but not limited to, CD7. In some embodiments, the targeting moiety has a sequence as set forth in SEQ ID NO: 2. In some embodiments, the Ebola virus glycoprotein comprises an insertion of the targeting moiety amino acid sequence as set forth in SEQ ID NO: 2 in place of the glycan cap amino acid sequence, a mucin-like domain (MLD) amino acid sequence, or any combination thereof. In some embodiments, the mutation comprises an insertion of the sequence as set forth in SEQ ID NO: 2 in place of the glycan cap amino acid sequence. In some embodiments. the mutation comprises an insertion of the sequence as set forth in SEQ ID NO: 2 in place of the amino acid sequence from or between positions 213-306 as compared to SEQ ID NO: 1; an insertion of the sequence as set forth in SEQ ID NO: 2 in place of the amino acid sequence from or between positions 305-484 as compared to SEQ ID NO: 1; an insertion of the sequence as set forth in SEQ ID NO: 2 in place of the amino acid sequence from or between positions 213-484 as compared to SEQ ID NO: 1; an insertion of the sequence as set forth in SEQ ID NO: 2 in place of the amino acid sequence from or between positions 213-497 as compared to SEQ ID NO: 1; or an insertion of the sequence as set forth in SEQ ID NO: 2 in place of the amino acid sequence from or between positions 232-497 as compared to SEQ ID NO: 1. In some embodiments, the insertion of the sequence as set forth in SEQ ID NO: 2 in place of the glycan cap amino acid sequence comprises an insertion from or between amino acid residues at positions 213 and 306 as compared to SEQ ID NO: 1. In some embodiments, the insertion of the sequence as set forth in SEQ ID NO: 2 in place of the glycan cap amino acid sequence comprises an insertion from or between amino acid residues at positions 232 and 306 as compared to SEQ ID NO: 1. In some embodiments, the mutation comprises an insertion of the sequence as set forth in SEQ ID NO: 2 in place of the MLD amino acid sequence. In some embodiments, the insertion of the sequence as set forth in SEQ ID NO: 2 in place of the MLD amino acid sequence comprises an insertion from or between amino acid residues at positions 305 and 497 as compared to SEQ ID NO: 1. In some embodiments, the insertion of the sequence as set forth in SEQ ID NO: 2 in place of the MLD amino acid sequence comprises an insertion from or between amino acid residues at positions 305 and 484 as compared to SEQ ID NO: 1. In some embodiments, the mutation comprises an insertion of the sequence as set forth in SEQ ID NO: 2 in place of the glycan cap amino acid sequence and the MLD amino acid sequence. In some embodiments, the insertion of the sequence as set forth in SEQ ID NO: 2 in place of the glycan cap amino acid sequence and the MLD amino acid sequence comprises an insertion from or between amino acid residues at positions 213 and 484 as compared to SEQ ID NO: 1. In some embodiments, the insertion of the sequence as set forth in SEQ ID NO: 2 in place of the glycan cap amino acid sequence and the MLD amino acid sequence comprises an insertion from or between amino acid residues at positions 213 and 497 as compared to SEQ ID NO: 1. In some embodiments, the insertion of the sequence as set forth in SEQ ID NO: 2 in place of the glycan cap amino acid sequence and the MLD amino acid sequence comprises an insertion from or between amino acid residues at positions 232 and 497 as compared to SEQ ID NO: 1. In some embodiments, the Ebola virus glycoprotein has a sequence as set forth in SEQ ID NO: 5, 6, 7, 8, or 9.

EBOVΔCap (anti-CD8 DARPin)
(SEQ ID NO: 5)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYSAGGGGSGGGGSGGGGSASDLGKKLLEASRAGQDDEVRILMANGADV
NAQDRYGTTPLHLAAWHGHLEIVEVLLKHGADVNANDVKGNTPLHLAANVGHLEIVEVLLKYGAD
VNAADNWGHTPLHLAAFWGHLEIVEVLLKYGADVNAQDKFGKTPFDLAIDNGNEDIAEVLQKAAT
GGGGGSGGGGSGGGGSSALSFTVVSNGAKNISGQSPARTSSDPGTNTTTEDHKIMASENSSAMVQ
VHSQGREAAVSHLTTLATISTSPQSLTTKPGPDNSTHNTPVYKLDISEATQVEQHHRRTDNDSTA
SDTPSATTAAGPPKAENTNTSKSTDFLDPATTTSPQNHSETAGNNNTHHQDTGEESASSGKLGLI
TNTIAGVAGLITGGRRTRREAIVNAQPKCNPNLHYWTTQDEGAAIGLAWIPYFGPAAEGIYIEGL
MHNQDGLICGLRQLANETTQALQLFLRATTELRTFSILNRKAIDFLLQRWGGTCHILGPDCCIEP
HDWTKNITDKIDQIIHDFVDKTLPDQGDNDNWWTGWRQWIPAGIGVTGVIIAVIALFCICKFVF;
EBOVΔMLD (anti-CD8 DARPin)
(SEQ ID NO: 6)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEESAGGGGSGGGGSGGGGSASD
LGKKLLEASRAGQDDEVRILMANGADVNAQDRYGTTPLHLAAWHGHLEIVEVLLKHGADVNANDV
KGNTPLHLAANVGHLEIVEVLLKYGADVNAADNWGHTPLHLAAFWGHLEIVEVLLKYGADVNAQD
KFGKTPFDLAIDNGNEDIAEVLQKAATGGGGGSGGGGSGGGGSSANTIAGVAGLITGGRRTRREA
IVNAQPKCNPNLHYWTTQDEGAAIGLAWIPYFGPAAEGIYIEGLMHNQDGLICGLRQLANETTQA
LQLFLRATTELRTFSILNRKAIDFLLQRWGGTCHILGPDCCIEPHDWTKNITDKIDQIIHDFVDK
TLPDQGDNDNWWTGWRQWIPAGIGVTGVIIAVIALFCICKFVF;
EBOVΔCap + MLD (anti-CD8 DARPin)
(SEQ ID NO: 7)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYSAGGGGSGGGGSGGGGSASDLGKKLLEASRAGQDDEVRILMANGADV
NAQDRYGTTPLHLAAWHGHLEIVEVLLKHGADVNANDVKGNTPLHLAANVGHLEIVEVLLKYGAD
VNAADNWGHTPLHLAAFWGHLEIVEVLLKYGADVNAQDKFGKTPFDLAIDNGNEDIAEVLQKAAT
GGGGGSGGGGSGGGGSSANTIAGVAGLITGGRRTRREAIVNAQPKCNPNLHYWTTQDEGAAIGLA
WIPYFGPAAEGIYIEGLMHNQDGLICGLRQLANETTQALQLFLRATTELRTFSILNRKAIDFLLQ
RWGGTCHILGPDCCIEPHDWTKNITDKIDQIIHDFVDKTLPDQGDNDNWWTGWRQWIPAGIGVTG
VIIAVIALFCICKFVF;
EBOVΔCap + MLDf
(SEQ ID NO: 8)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYSAGGGGSGGGGSGGGGSASDLGKKLLEASRAGQDDEVRILMANGADV
NAQDRYGTTPLHLAAWHGHLEIVEVLLKHGADVNANDVKGNTPLHLAANVGHLEIVEVLLKYGAD
VNAADNWGHTPLHLAAFWGHLEIVEVLLKYGADVNAQDKFGKTPFDLAIDNGNEDIAEVLQKAAT
GGGGGSGGGGSGGGGSSARRTRREAIVNAQPKCNPNLHYWTTQDEGAAIGLAWIPYFGPAAEGIY
IEGLMHNQDGLICGLRQLANETTQALQLFLRATTELRTFSILNRKAIDFLLQRWGGTCHILGPDC
CIEPHDWTKNITDKIDQIIHDFVDKTLPDQGDNDNWWTGWRQWIPAGIGVTGVIIAVIALFCICK
FVF;
or
EBOVΔCap233 + MLD
(SEQ ID NO: 9)
MGVTGILQLPRDRFKRTSFFLWVIILFQRTFSIPLGVIHNSTLQVSDVDKLVCRDKLSSTNQLRS
VGLNLEGNGVATDVPSATKRWGFRSGVPPKVVNYEAGEWAENCYNLEIKKPDGSECLPAAPDGIR
GFPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYSAGGGGSGGGGSGGGGSASDLGKKLLEA
SRAGQDDEVRILMANGADVNAQDRYGTTPLHLAAWHGHLEIVEVLLKHGADVNANDVKGNTPLHL
AANVGHLEIVEVLLKYGADVNAADNWGHTPLHLAAFWGHLEIVEVLLKYGADVNAQDKFGKTPFD
LAIDNGNEDIAEVLQKAATGGGGGSGGGGSGGGGSSARRTRREAIVNAQPKCNPNLHYWTTQDEG
AAIGLAWIPYFGPAAEGIYIEGLMHNQDGLICGLRQLANETTQALQLFLRATTELRTFSILNRKA
IDFLLQRWGGTCHILGPDCCIEPHDWTKNITDKIDQIIHDFVDKTLPDQGDNDNWWTGWRQWIPA
GIGVTGVIIAVIALFCICKFVF.

### Cells

In some embodiments, the embodiments provided include a cell comprising the engineered virus particle or particles as provided herein. In some embodiments, the cell comprises the polypeptide of interest encoded by the engineered virus particle.

In some embodiments, the polypeptide of interest is a hemoglobin beta chain. In some embodiments, the polypeptide of interest is a heterologous chimeric antigen receptor (CAR).

In some embodiments, the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, any number of T cell lines available in the art, may be used.

In some embodiments, a cell comprising the polypeptide of interest encoded for by the engineered viral particle as described herein is provided. In some embodiments, the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

### Viral Vector

Provided herein are compositions that can be used, for example, to selectively transduce a cell expressing specific cell surface markers in vivo using a viral vector. In some embodiments, the vector encodes the polypeptide of interest. In some embodiments, the viral vector is a pseudotyped vector.

Expression vectors comprising a nucleic acid of the present disclosure can be introduced into a host cell by any method or composition known to persons skilled in the art. The expression vectors may include viral sequences for transfection, if desired. Alternatively, the expression vectors may be introduced by fusion, electroporation, biolistics, transfection, lipofection, or the like. Although the cell can be transduced or transfected in vivo, in some embodiments, the transduced cells can then be isolated from the subject and then, in some embodiments, may be grown and expanded in culture ex vivo. The expanded cells can then be screened by virtue of a marker present in the vectors. The expanded cells can then be reintroduced into the same subject or a different subject for treatment. Various markers that may be used are known in the art, and may include hprt, neomycin resistance, thymidine kinase, hygromycin resistance, etc. In some embodiments, the host cell is an immune cell or precursor thereof, e.g., a T cell, an NK cell, or an NKT cell.

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the vectors should not be limited to the use of constitutive promoters. Inducible promoters can also be used. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

Expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viruses, which are useful as vectors include, but are not limited to, retroviruses, herpes viruses, and lentiviruses.

In some embodiments, an expression vector (e.g., a lentiviral vector) may be used to introduce the polypeptide of interest (e.g., CAR, or a portion thereof) into a cell (e.g., a T cell). Accordingly, an expression vector (e.g., a lentiviral vector) may comprise a nucleic acid encoding for a polypeptide of interest (e.g., CAR, or a portion thereof). As provided herein, the polypeptide of interest can be introduced into the cell through the use of expression vectors.

### Methods of Treatment

Also provided herein are methods of treating a disease in a subject in need thereof.

In some embodiments, the methods provided include, but are not limited to, methods of treating a disease in a subject in need thereof, comprising administering to the subject the viral particle(s) provided herein to treat the disease.

In some embodiments, the disease is a cancer. In addition, the compositions provided for herein can be used in methods for the treatment of any condition related to a cancer, such as a cell-mediated immune response against a tumor cell(s), where it is desirable to treat or alleviate the disease. The types of cancers to be treated include, but are not limited to, carcinoma, blastoma, sarcoma, certain leukemia or lymphoid malignancies, such as, but not limited to, adult acute lymphoblastic leukemia, chronic lymphocytic leukemia, follicular lymphoma, mantle cell lymphoma, Burkitt lymphoma, benign and malignant tumors, malignancies e.g., sarcomas, carcinomas, and melanomas. Other exemplary cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma and brain metastases).

Carcinomas that can be amenable to therapy by the methods disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In some embodiments, the compositions provided herein can be used in methods to treat a myeloma, or a condition related to myeloma. Examples of myeloma or conditions related thereto include, without limitation, light chain myeloma, non-secretory myeloma, monoclonal gamopathy of undetermined significance (MGUS), plasmacytoma (e.g., solitary, multiple solitary, extramedullary plasmacytoma), amyloidosis, and multiple myeloma. In some embodiments, methods of treating multiple myeloma are provided. In some embodiments, the multiple myeloma is refractory myeloma. In some embodiments, the multiple myeloma is relapsed myeloma.

In some embodiments, the in vivo modified immune cells produced using the vectors and compositions provided herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In some embodiments, the melanoma is cutaneous melanoma. In some embodiments, the melanoma is refractory melanoma. In some embodiments, the melanoma is relapsed melanoma.

In some embodiments, the vectors and compositions provided herein are used to treat a sarcoma, or a condition related to sarcoma. Examples of sarcoma or conditions related thereto include, without limitation, angiosarcoma, chondrosarcoma, chordoma, endotheliosarcoma, Ewing's sarcoma, fibrosarcoma, gastrointestinal stromal tumor, leiomyosarcoma, liposarcoma, lymphangiosarcoma, lymphangioendotheliosarcoma, mesothelioma, malignant peripheral nerve sheath tumor, myxosarcoma, osteogenic sarcoma, osteosarcoma, pleomorphic sarcoma, rhabdomyosarcoma, synovioma, synovial sarcoma, and other soft tissue sarcomas. In some embodiments, the sarcoma is synovial sarcoma. In some embodiments, the sarcoma is liposarcoma such as myxoid/round cell liposarcoma, differentiated/dedifferentiated liposarcoma, or pleomorphic liposarcoma. In some embodiments, the sarcoma is myxoid/round cell liposarcoma. In some embodiments, the sarcoma is refractory sarcoma. In some embodiments, the sarcoma is relapsed sarcoma.

In some embodiments, the compositions and vectors are used in methods for treating sickle cell disease. In some embodiments, the viral vectors or particles are administered to a subject suffering from sickle cell disease, wherein the particles encode the hemoglobin beta chain. When expressed in the cell, the hemoglobin beta chain is expressed and alleviates the symptoms of sickle cell disease to treat the disease.

In some embodiments, the subject has been treated with a therapeutic agent targeting the disease or condition, e.g. the tumor, prior to administration of the composition or plurality of viral vectors. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

The administration of the compositions may be carried out in any convenient manner known to those of skill in the art. For example, the compositions may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the composition is injected directly into a site of a local disease site in the subject, a lymph node, an organ, a tumor, and the like.

For the prevention or treatment of disease, the appropriate dosage may depend on the type of disease to be treated, the severity and course of the disease, whether the composition is administered for preventive or therapeutic purposes, previous therapy, the subject's clinical history and response to the treatment, and the discretion of the attending physician. The composition is, in some embodiments, suitably administered to the subject at one time or over a series of treatments.

In some embodiments, the composition is administered as part of a combination treatment, such as simultaneously with or sequentially with, in any order, another therapeutic intervention, such as an antibody or engineered cell or receptor or agent, such as a cytotoxic or therapeutic agent. The composition(s), in some embodiments, is co-administered with one or more additional therapeutic agents or in connection with another therapeutic intervention, either simultaneously or sequentially in any order. In some contexts, the composition is co-administered with another therapy sufficiently close in time such that the composition enhances the effect of one or more additional therapeutic agents, or vice versa. In some embodiments, the composition is administered prior to the one or more additional therapeutic agents. In some embodiments, the composition is administered after the one or more additional therapeutic agents. In some embodiments, the one or more additional agents includes a cytokine, such as IL-2, for example, to enhance persistence. In some embodiments, the methods comprise administration of a chemotherapeutic agent. In some embodiments, the methods do not comprise the administration of a chemotherapeutic agent.

In some embodiments, the compositions may be administered to a subject in combination with an immune checkpoint antibody (e.g., an anti-PD1, anti-CTLA-4, or anti-PDL1 antibody). For example, viral vectors may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In some embodiments, the compositions may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In some embodiments, the composition may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the viral vector. In some embodiments, combination treatment comprising an immune checkpoint modulator may increase the therapeutic efficacy of a therapy comprising a composition as provided herein. The other therapeutic can be administered simultaneously, before, or after the vectors provided herein are administered to the subject.

In some embodiments, the subject is provided a secondary treatment. Secondary treatments include but are not limited to chemotherapy, radiation, surgery, and medications. In some embodiments, the subject is not provided a secondary treatment.

In some embodiments, methods of treating cancer in a subject in need thereof are provided, the methods comprising administering to the subject any of the compositions, such as the viral particle(s), provided herein. In some embodiments methods of treating cancer in a subject in need thereof are provided, the methods comprising administering to the subject a composition generated by any one of the methods disclosed herein.

In some embodiments, a method of in vivo delivery of a molecule of interest to a target cell, the method comprising administering an engineered viral particle to a subject in need of delivery, wherein the engineered viral particle comprises an engineered envelope comprising a recombinant Ebola virus glycoprotein, or a variant thereof, a gag-pol protein, and an engineered targeting moiety for binding to the target cell; a nucleic acid encoding a the molecule of interest; and wherein the administration of the engineered viral particle delivers the nucleic acid molecule encoding the molecule of interest to the cell is provided.

In some embodiments, a method of in vivo delivery of a molecule of interest to a target cell, the method comprising administering an engineered viral particle to a subject in need of delivery, wherein the engineered viral particle comprises an engineered envelope comprising an Ebola virus glycoprotein fused or linked to a targeting moiety, and a gag-pol protein, wherein the Ebola virus glycoprotein fused or linked to the targeting moiety has the amino acid sequence as set forth in SEQ ID NO: 5-9; a nucleic acid encoding a molecule of interest; and wherein the administration of the engineered viral particle delivers the nucleic acid molecule encoding the molecule of interest to the cell is provided.

In some embodiments, a method of delivering a peptide of interest to a target cell, such as an antigen presenting cell (“APC”), the method comprising contacting an engineered viral particle, as described herein, for binding to a target cell with an APC, thereby delivering the peptide of interest to the APC is provided.

### Pharmaceutical Compositions and Formulations

The compositions disclosed herein can comprise a pharmaceutical composition, and for example include a pharmaceutically acceptable carrier, and/or a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some aspects, buffering agents are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition. In some embodiments, the pharmaceutical composition does not include a chemotherapeutic.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described some embodiments in detail, the same will be more clearly understood by reference to the following examples, which are included for purposes of illustration only and are not intended to be limiting.

In some embodiments, a pharmaceutical composition comprising a cell as described herein is provided. In some embodiments, a pharmaceutical composition comprising an engineered viral particle, as described herein, bound to a cell is provided. In some embodiments, the cell is T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+. In some embodiments, the cell is an isolated cell. In some embodiments, a pharmaceutical composition comprising an engineered viral vector as described herein is provided.

### Enumerated List of Embodiments

1. An engineered viral particle comprising:
   i. an engineered envelope comprising a recombinant Ebola virus glycoprotein, or a variant thereof, a gag-pol protein, and an engineered targeting moiety for binding to a target cell; and
   ii. a nucleic acid encoding a polypeptide of interest.
2. The engineered viral particle of embodiment 1, wherein the targeting moiety is fused to the Ebola virus glycoprotein.
3. The engineered viral particle of any one of embodiments 1 or 2, wherein the viral particle is a lentivirus.
4. The engineered viral particle of any one of embodiments 1-3, wherein the viral particle is pseudotyped with the recombinant Ebola virus glycoprotein.
5. The engineered viral particle of embodiment 4, wherein the pseudotyped viral particle is a pseudotyped lentivirus.
6. The engineered viral particle of any one of embodiments 1-5, wherein the Ebola virus glycoprotein comprises a sequence comprising a deletion, a mutation, or any combination thereof as compared to SEQ ID NO: 1.
7. The engineered viral particle of embodiment 6, wherein the deletion comprises an amino acid deletion of a glycan cap amino acid sequence, a mucin-like domain (MLD) amino acid sequence, or any combination thereof.
8. The engineered viral particle of any one of embodiments 6 or 7, wherein the deletion comprises an amino acid deletion of a glycan cap amino acid sequence.
9. The engineered viral particle of any one of embodiments 6 or 7, wherein the deletion comprises an amino acid deletion of a MLD amino acid sequence.
10. The engineered viral particle of any one of embodiments 6-9, wherein the deletion comprises deletion of a glycan cap sequence, a MLD amino acid sequence, or any combination thereof, from or between positions 213-306, 305-484, 213-484, 213-497, or 232-497, as compared to SEQ ID NO: 1.
11. The engineered viral particle of any one of embodiments 6-10, wherein the deletion comprises deletion of a glycan cap sequence from or between positions 213-306, or 232-306, as compared to SEQ ID NO: 1.
12. The engineered viral particle of any one of embodiments 6-10, wherein the deletion comprises deletion of a MLD amino acid sequence from or between positions 305-484, or 305-497, as compared to SEQ ID NO: 1.
13. The engineered viral particle of any one of embodiments 6-10, wherein the deletion comprises deletion of a glycan cap amino acid sequence, a MLD amino acid sequence, or any combination thereof, from or between positions 213-484, 213-497, or 232-497, as compared to SEQ ID NO: 1.
14. The engineered viral particle of any one of embodiments 6-13, wherein the deletion comprises an amino acid deletion from or between positions 213-306, 305-484, 213-484, 213-497, or 232-497, as compared to SEQ ID NO: 1.
15. The engineered viral particle of any one of embodiments 6-14, wherein the deletion comprises an amino acid deletion at any position from or between positions 213-306, 305-484, 213-484, 213-497, or 232-497, as compared to SEQ ID NO: 1.
16. The engineered viral particle of embodiment 6, wherein the mutation comprises an insertion of a targeting moiety amino acid sequence in place of the glycan cap amino acid sequence, a MLD amino acid sequence, or any combination thereof.
17. The engineered viral particle of any one of embodiments 6 or 16, wherein the mutation comprises an amino acid insertion at any position from or between positions 213-306, 305-484, 213-484, 213-497, or 232-497, as compared to SEQ ID NO: 1.
18. The engineered viral particle of any one of embodiments 6 or 16-17, wherein the mutation comprises:
   an insertion of a targeting moiety amino acid sequence in place of the amino acid sequence from or between positions 213-306 as compared to SEQ ID NO: 1;
   an insertion of a targeting moiety amino acid sequence in place of the amino acid sequence from or between positions 305-484 as compared to SEQ ID NO: 1;
   an insertion of a targeting moiety amino acid sequence in place of the amino acid sequence from or between positions 213-484 as compared to SEQ ID NO: 1;
   an insertion of a targeting moiety amino acid sequence in place of the amino acid sequence from or between positions 213-497 as compared to SEQ ID NO: 1; or
   an insertion of a targeting moiety amino acid sequence in place of the amino acid sequence from or between positions 232-497 as compared to SEQ ID NO: 1.
19. The engineered viral particle of any one of embodiments 1-18, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
20. The engineered viral particle of any one of embodiments 1-19, wherein the targeting moiety is fused to the Ebola virus glycoprotein via a linker, such as a peptide linker.
21. The engineered viral particle of embodiment 20, wherein the linker has a sequence as set forth in SEQ ID NO: 3 or 4.
22. The engineered viral particle of any one of embodiments 1-21, wherein the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.
23. The engineered viral particle of any one of embodiments 1-22, wherein the targeting moiety is a CD8 binding moiety.
24. The engineered viral particle of embodiment 23, wherein the CD8 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
25. The engineered viral particle of any one of embodiments 23 or 24, wherein the CD8 binding moiety is an anti-CD8 DARPin.
26. The engineered viral particle of embodiment 25, wherein the anti-CD8 DARPin comprises a sequence as set forth in SEQ ID NO: 2.
27. The engineered viral particle of any one of embodiments 1-22, wherein the targeting moiety is a CD7 binding moiety.
28. The engineered viral particle of embodiment 27, wherein the CD7 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
29. The engineered viral particle of any one of embodiments 27 or 28, wherein the CD7 binding moiety is an anti-CD7 DARPin.
30. An engineered viral particle comprising:
   i. an engineered envelope comprising an Ebola virus glycoprotein fused or linked to a targeting moiety, and a gag-pol protein, wherein the Ebola virus glycoprotein fused or linked to a targeting moiety has the amino acid sequence as set forth in SEQ ID NO: 5-9; and
   ii. a nucleic acid encoding a polypeptide of interest.
31. The engineered viral particle of embodiment 30, wherein the viral particle is a lentivirus.
32. The engineered viral particle of any one of embodiments 30 or 31, wherein the lentivirus is pseudotyped with the Ebola virus glycoprotein.
33. The engineered viral particle of any one of embodiments 1-32, wherein the polypeptide of interest is a chimeric antigen receptor (CAR), an antigen, an enzyme, a protein, such as a hemoglobin beta chain.
34. A polypeptide molecule comprising a targeting moiety fused or linked to an Ebola virus glycoprotein in place of the glycan cap, and/or the MLD of the Ebola virus glycoprotein.
35. The polypeptide of embodiment 34, wherein the targeting moiety is fused or linked to the Ebola virus glycoprotein via a linker, such as a peptide linker.
36. The polypeptide of any one of embodiments 34 or 35, wherein the linker is a glycine/serine linker.
37. The polypeptide of any one of embodiments 34-36, wherein the linker has a sequence as set forth in SEQ ID NO: 3 or 4.
38. The polypeptide of any one of embodiments 34-37, wherein the targeting moiety is fused to the Ebola virus glycoprotein at a position from or between positions 213-306, 305-484, 213-484, 213-497, and 232-497, as compared to SEQ ID NO: 1.
39. The polypeptide of any one of embodiments 34-38, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
40. The polypeptide of any one of embodiments 34-39, wherein the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.
41. The polypeptide of any one of embodiments 34-40, wherein the targeting moiety is a CD8 binding moiety.
42. The polypeptide of embodiment 41, wherein the CD8 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
43. The polypeptide of any one of embodiments 37 or 38, wherein the CD8 binding moiety is an anti-CD8 DARPin.
44. The polypeptide of embodiment 39, wherein the anti-CD8 DARPin comprises a sequence as set forth in SEQ ID NO: 2.
45. The polypeptide of any one of embodiments 34-40, wherein the targeting moiety is a CD7 binding moiety.
46. The polypeptide of embodiment 45, wherein the CD7 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
47. The polypeptide of any one of embodiments 45 or 46, wherein the CD7 binding moiety is an anti-CD7 DARPin.
48. A polypeptide molecule comprising an EBOV GP, or a variant thereof, fused to a targeting moiety via a linker, such as a peptide linker, and having a sequence as set forth in SEQ ID NO: 5-9.
49. A method of in vivo delivery of a molecule of interest to a target cell, the method comprising administering an engineered viral particle to a subject in need of delivery, wherein the engineered viral particle comprises:
   an engineered envelope comprising a recombinant Ebola virus glycoprotein, or a variant thereof, a gag-pol protein, and an engineered targeting moiety for binding to the target cell;
   a nucleic acid encoding the molecule of interest; and
      wherein the administration of the engineered viral particle delivers the nucleic acid molecule encoding the molecule of interest to the cell.
50. The method of embodiment 49, wherein the viral particle is pseudotyped with the Ebola virus glycoprotein.
51. The method of embodiment 50, wherein the pseudotyped viral particle is a pseudotyped lentivirus.
52. The method of embodiment 51, wherein the Ebola virus glycoprotein comprises a sequence comprising a deletion, a mutation, or any combination thereof as compared to SEQ ID NO: 1.
53. The method of embodiment 52, wherein the deletion comprises an amino acid deletion of a glycan cap amino acid sequence, a MLD amino acid sequence, or any combination thereof.
54. The method of any one of embodiments 52 or 53, wherein the deletion comprises an amino acid deletion of a glycan cap amino acid sequence.
55. The method of any one of embodiments 52-54, wherein the deletion comprises an amino acid deletion of a MLD amino acid sequence.
56. The method of any one of embodiments 52-55, wherein the deletion comprises deletion of a glycan cap sequence, a MLD amino acid sequence, or any combination thereof, from or between positions 213-306, 305-484, 213-484, 213-497, or 232-497, as compared to SEQ ID NO: 1.
57. The method of any one of embodiments 52-56, wherein the deletion comprises deletion of a glycan cap sequence from or between positions 213-306, or 232-306, as compared to SEQ ID NO: 1.
58. The method of any one of embodiments 52-57, wherein the deletion comprises deletion of a MLD amino acid sequence from or between positions 305-484, or 305-497, as compared to SEQ ID NO: 1.
59. The method of any one of embodiments 52-58, wherein the deletion comprises deletion of a glycan cap amino acid sequence, a MLD amino acid sequence, or any combination thereof, from or between positions 213-484, 213-497, or 232-497, as compared to SEQ ID NO: 1.
60. The method of any one of embodiments 52-59, wherein the deletion comprises an amino acid deletion from or between positions 213-306, 305-484, 213-484, 213-497, or 232-497, as compared to SEQ ID NO: 1.
61. The method of any one of embodiments 52-60, wherein the deletion comprises an amino acid deletion at any position from or between positions 213-306, 305-484, 213-484, 213-497, or 232-497, as compared to SEQ ID NO: 1.
62. The method of embodiment 52, wherein the mutation comprises an insertion of a targeting moiety amino acid sequence in place of the glycan cap amino acid sequence, a MLD amino acid sequence, or any combination thereof.
63. The method of any one of embodiments 52 or 62, wherein the mutation comprises an insertion of a targeting moiety amino acid sequence in place of the glycan cap amino acid sequence, a MLD amino acid sequence, or any combination thereof, from or between positions 213-306, 305-484, 213-484, 213-497, or 232-497, as compared to SEQ ID NO: 1.
64. The method of any one of embodiments 52 or 63, wherein the mutation comprises:
   an insertion of a targeting moiety amino acid sequence in place of the amino acid sequence from or between positions 213-306 as compared to SEQ ID NO: 1;
   an insertion of a targeting moiety amino acid sequence in place of the amino acid sequence from or between positions 305-484 as compared to SEQ ID NO: 1;
   an insertion of a targeting moiety amino acid sequence in place of the amino acid sequence from or between positions 213-484 as compared to SEQ ID NO: 1;
   an insertion of a targeting moiety amino acid sequence in place of the amino acid sequence from or between positions 213-497 as compared to SEQ ID NO: 1; or
   an insertion of a targeting moiety amino acid sequence in place of the amino acid sequence from or between positions 232-497 as compared to SEQ ID NO: 1.
65. The method of embodiment 52, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.
66. A method of in vivo delivery of a molecule of interest to a target cell, the method comprising administering an engineered viral particle to a subject in need of delivery, wherein the engineered viral particle comprises:
   an engineered envelope comprising an Ebola virus glycoprotein fused or linked to a targeting moiety, and a gag-pol protein, wherein the Ebola virus glycoprotein fused or linked to the targeting moiety has the amino acid sequence as set forth in SEQ ID NO: 5-9;
   a nucleic acid encoding a molecule of interest; and
      wherein the administration of the engineered viral particle delivers the nucleic acid molecule encoding the molecule of interest to the cell.
67. The method of embodiment 66, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.
68. A method of delivering a peptide of interest to a target cell, such as an antigen presenting cell (“APC”), the method comprising:
   contacting an engineered viral particle of any one of embodiments 1-29 for binding to a target cell with an APC, thereby delivering the peptide of interest to the APC.
69. The method of embodiment 68, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.
70. A cell comprising the polypeptide of interest encoded for by the engineered viral particle of any one of embodiments 1-33.
71. The cell of embodiment 70, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.
72. A pharmaceutical composition comprising the cell of embodiment 70.
73. A composition comprising the engineered viral particle of any one of embodiments 1-33 bound to a cell.
74. The composition of embodiment 73, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.
75. The composition of any one of embodiments 73 or 74, wherein the cell is an isolated cell.
76. A pharmaceutical composition comprising the engineered viral particle of any one of embodiments 1-33.
77. A method of treating a disease in a subject, the method comprising administering to the subject the engineered viral particle of any one of embodiments 1-33, wherein the engineered viral particle expresses the polypeptide of interest in a cell.
78. The method of embodiment 77, wherein the disease is as provided herein.
79. The method of embodiments 77 or 78, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

### EXAMPLES

The embodiments are now described with reference to the following Examples. These Examples are provided for the purpose of illustration only, and the embodiments are not limited to these Examples, but rather encompasses all variations that are evident as a result of the teachings provided herein.

### Example 1: Generation of Plasmids and Sequences

All Ebola virus sequences are based on the reference strain Ebola Zaire Mayinga 1976 (accession NC002549). Ebola sequences were synthesized by GenScript (Piscataway, NJ) using codon optimization for human expression. Deletions of various Ebola virus glycoprotein amino acids are provided herein. Proteins that bind to specific cellular targets were inserted in the deleted regions of the Ebola virus glycoprotein and flanked by a glycine-serine linker (G4S). Example of a protein that binds to a specific target that was used, but is not limited to, was a DARPIN binding to human CD8 (Dp53F6 sequence from PMID: 32160795 DOI: 10.1089/hum.2019.248). Any binding domain can be inserted as the targeting moiety. The resulting recombinant Ebola GPs were expressed under the direction of a CMV promoter.

### Example 2: Generation of Engineered Lentiviral Particles

The recombinant lentiviral particles expressing the Ebola virus glycoprotein incorporated on the surface were generated by plasmid transection into HEK293T cells using Lipofectamine 3000 (ThermoFisher Scientific). A total of 4 plasmids were transfected: (1) plasmid expressing the Ebola virus glycoprotein, (2) lentiviral genome expressing eGFP, (3) plasmid expressing gag-pol, (4) plasmid expressing rev. Media was changed 6 hours after transfection and cells were harvested 48 hours later. After transfection, the HEK293T producer cells were stained with an anti-Ebola GP antibody (Absolute Antibody, Wilton, UK, 00690-23.0, clone KZ52) to confirm the Ebola GP is expressed on the cell surface. Virus in the media was concentrated by centrifugation through a sucrose cushion and resuspended in PBS. Lentiviral particle titer was determined using the Lenti-X p24 Rapid Titer Kit (Takara Bio, San Jose, CA)

### Example 3: Ebola Virus Glycoprotein With DARPin Inserted in Place of MLD is Expressed in a Native-Like Conformation

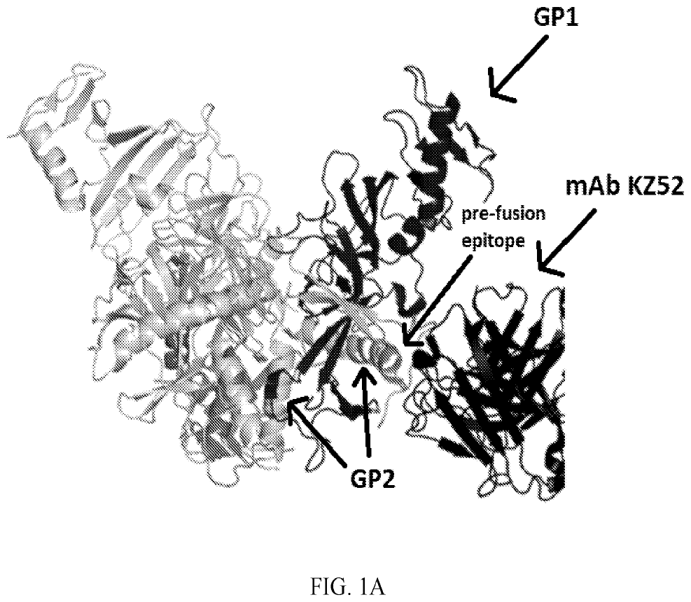

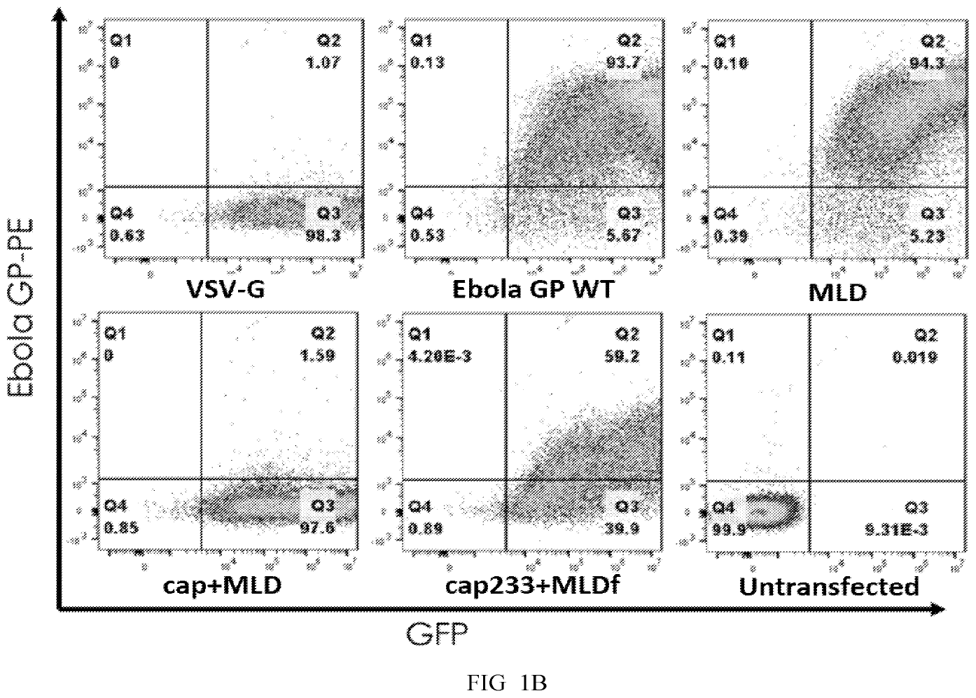

HEK293T cells were grown in DMEM with 10% FBS. SupT1 cells were maintained in RPMI media with 10% FBS. A series of 3-fold dilutions (in cell culture media) of the concentrated lentivirus was performed and used to infect HEK293T cells and SupT1. Media was replaced 24 hours later, and the transduced cells were analyzed by flow cytometry on days 4 and 7 after transduction. Cells were stained with an anti-CD8 antibody to detect CD8 positive cells (BV421 mouse-anti-human CD8, clone RPA-T8, BD Biosciences) as well as GFP expression. Cell viability was also determined. Immediately following harvesting virus-containing supernatant, transfected 293T cells were resuspended and stained using the anti-Ebola virus glycoprotein monoclonal antibody KZ52. This neutralizing antibody recognizes a conformational epitope found only in the pre-fusion trimer, thus it serves as a sensitive indicator of properly folded glycoprotein (PMID: 18615077) (FIG. 1A). As expected, cells expressing the wild type Ebola virus glycoprotein stained positive compared to cells expressing VSV-G (FIG. 1B). Cells transfected with the construct in which the CD8-specific DARPin dp53F6 was inserted in place of the MLD also stained positive, demonstrating expression and proper folding of this chimeric glycoprotein. In contrast, constructs in which this same DARPin was cloned into glycoprotein with various length of the glycan cap and MLD removed showed reduced staining. The glycan cap has been shown to fold over the NPC1 binding pocket in the base of glycoprotein to prevent premature triggering; consistent with this, retention of this part of the glycan cap (through position 232) increased glycoprotein staining.

### Example 4: Insertion of CD8-DARPin Confers Ebola Virus Glycoprotein With the Ability to Enter SupT1 Cells

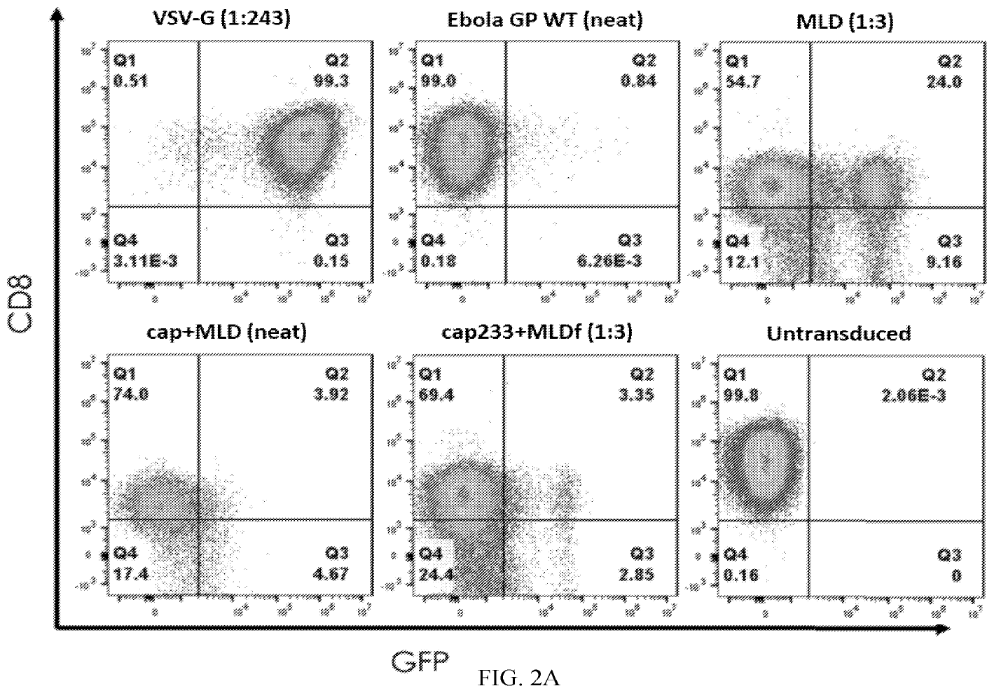

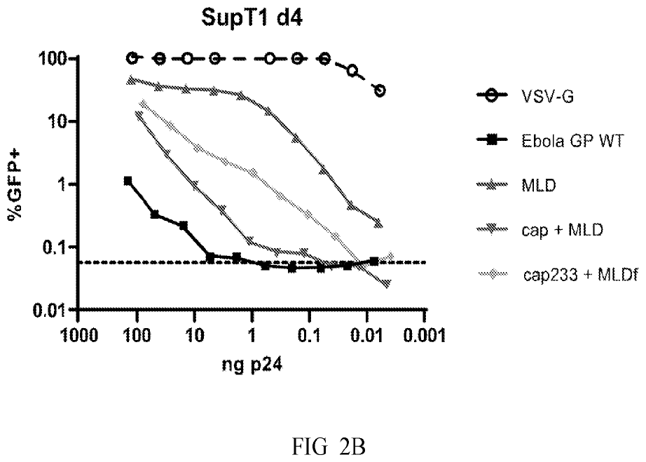

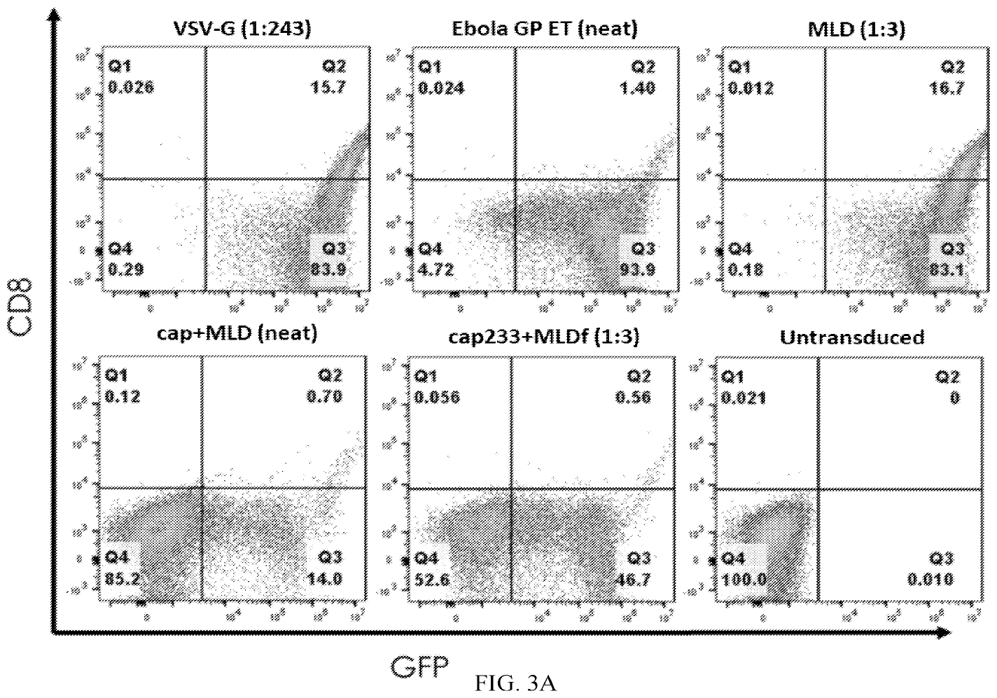

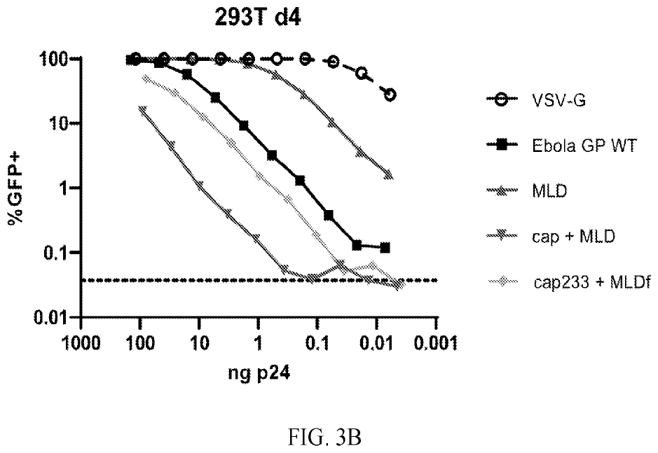

Insertion of CD8-DARPin confers Ebola virus glycoprotein with the ability to enter SupT1 cells. Lentivirus harvested from 293T cells was concentrated and purified through a sucrose cushion and dilutions of virus were used to transduce the CD8+ SupT1 cell line. GFP was quantified four days post-transduction via flow cytometry as a measure of transduction efficiency (FIG. 2). Whereas wild-type Ebola virus glycoprotein failed to infect or mediate transduction of these cells (FIG. 3, PMID: 10775638), the Ebola construct with the CD8-targeting DARPIN in place of the MLD showed efficient transduction. The rank-order of transduction efficiency was consistent with the amount of GP expression observed in FIG. 1.

### Example 5: Insertion of CD8-DARPin Does Not Abrogate Native Ebola Virus Glycoprotein Tropism

Insertion of CD8-DARPin does not abrogate native Ebola virus glycoprotein tropism. In parallel to the transduction of CD8+ SupT1 cells, the same virus preparations were used to transduce CD8-293T cells (FIG. 3). Wild-type Ebola virus glycoprotein efficiently transduced these cells, consistent with the known tropism of Ebola virus glycoprotein pseudotypes (PMID: 10775638). The MLD construct showed a marked enhancement over wild-type glycoprotein. However, this is unlikely to be driven by the CD8-DARPin as 293T cells are uniformly CD8 negative. Previous reports have demonstrated enhanced Ebola virus glycoprotein infectivity upon removal of the MLD (PMID: 14599811), thus an important control in the next experiment is an identical construct lacking the CD8-DARPin. However, the observed enhancement on SupT1 cells is unlikely to be mediated by the MLD removal alone, as the particle: infectivity ratio on 293T cells was only ˜17-fold higher for the MLD construct compared to ˜2000-fold higher on CD8+ SupT1 cells (Table 1).

TABLE 1
Particle infectivity of Ebola GP variants. The particle-to-
infectivity ratio is shown for each construct on both
SupT1 (“on-target”) and 293T (“off-target”) cells.
Transduction efficiency was measured both four and seven
days post-transduction, yielding similar results.

| | Infectivity d4 | | Infectivity d7 | |
|---|---|---|---|---|
| | (TU/particle) | | (TU/particle) | |
| | SupT1 | 293T | SupT1 | 293T |
| VSV-G | 8.7E−02 | 8.1E−02 | 8.3E−02 | 8.9E−02 |
| Ebola GP WT | 1.5E−07 | 1.2E−04 | 2.9E−08 | 1.4E−04 |
| MLD | 6.2E−04 | 4.1E−03 | 5.5E−04 | 2.4E−03 |
| cap + MLD | 2.0E−06 | 2.7E−06 | 3.2E−07 | 2.5E−06 |
| cap233 + | 4.5E−05 | 3.7E−05 | 3.7E−05 | 4.6E−05 |
| MLDf | | | | |

The examples described herein are exemplary in manner and are not intended, nor should they be used, to limit the scope of the embodiments. Each and every reference, publication, accession number, patent, document, etc., is hereby incorporated by reference in its entirety for its intended purpose.

This description is not limited to the particular processes, compositions, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

From the foregoing, it will be appreciated that various embodiments of the present disclosure have been described herein for purposes of illustration, and that various modification can be made without departing from the scope and spirit of the present disclosure. Accordingly, the various embodiments disclosed herein are not intended to be limiting.

What is claimed:

1. An engineered viral particle comprising:
i. an engineered envelope comprising a recombinant Ebola virus glycoprotein, or a variant thereof, a gag-pol protein, and an engineered targeting moiety for binding to a target cell; and
ii. a nucleic acid molecule encoding a polypeptide of interest.

2. The engineered viral particle of claim 1, wherein the targeting moiety is fused to the Ebola virus glycoprotein.

3. The engineered viral particle of any one of claim 1 or 2, wherein the viral particle is a lentivirus.

4. The engineered viral particle of any one of claims 1-3, wherein the viral particle is pseudotyped with the recombinant Ebola virus glycoprotein.

5. The engineered viral particle of claim 4, wherein the pseudotyped viral particle is a pseudotyped lentivirus.

6. The engineered viral particle of any one of claims 1-5, wherein the Ebola virus glycoprotein comprises a sequence comprising a deletion, a mutation, insertion, or any combination thereof, as compared to a polypeptide comprising an amino acid sequence of SEQ ID NO: 1.

7. The engineered viral particle of claim 6, wherein the deletion comprises an amino acid deletion of a glycan cap amino acid sequence, a mucin-like domain (MLD) amino acid sequence, or any combination thereof.

8. The engineered viral particle of any one of claim 6 or 7, wherein the deletion comprises an amino acid deletion of a glycan cap amino acid sequence.

9. The engineered viral particle of any one of claim 6 or 7, wherein the deletion comprises an amino acid deletion of a MLD amino acid sequence.

10. The engineered viral particle of any one of claims 6-9, wherein the deletion comprises a deletion of a glycan cap sequence, a MLD amino acid sequence, or any combination thereof, from or between positions 213-306, 305-484, 213-484, 213-497, or 232-497, as compared to a polypeptide comprising an amino acid sequence of SEQ ID NO: 1.

11. The engineered viral particle of any one of claims 6-10, wherein the deletion comprises deletion of a glycan cap sequence from or between positions 213-306, or 232-306, as compared to a polypeptide comprising an amino acid sequence of SEQ ID NO: 1.

12. The engineered viral particle of any one of claims 6-10, wherein the deletion comprises deletion of a MLD amino acid sequence from or between positions 305-484, or 305-497, as compared to a polypeptide comprising an amino acid sequence of SEQ ID NO: 1.

13. The engineered viral particle of any one of claims 6-10, wherein the deletion comprises deletion of a glycan cap amino acid sequence, a MLD amino acid sequence, or any combination thereof, from or between positions 213-484, 213-497, or 232-497, as compared to a polypeptide comprising an amino acid sequence of SEQ ID NO: 1.

14. The engineered viral particle of any one of claims 6-13, wherein the deletion comprises an amino acid deletion from or between positions 213-306, 305-484, 213-484, 213-497, or 232-497, as compared to a polypeptide comprising an amino acid sequence of SEQ ID NO: 1.

15. The engineered viral particle of any one of claims 6-14, wherein the deletion comprises an amino acid deletion at any position from or between positions 213-306, 305-484, 213-484, 213-497, or 232-497, as compared to a polypeptide comprising an amino acid sequence of SEQ ID NO: 1.

16. The engineered viral particle of claim 6, wherein the mutation comprises an insertion of a targeting moiety amino acid sequence in place of the glycan cap amino acid sequence, a MLD amino acid sequence, or any combination thereof.

17. The engineered viral particle of any one of claim 6 or 16, wherein the mutation comprises an amino acid insertion at any position from or between positions 213-306, 305-484, 213-484, 213-497, or 232-497, as compared to a polypeptide comprising an amino acid sequence of SEQ ID NO: 1.

18. The engineered viral particle of any one of claim 6 or 16-17, wherein the mutation comprises:
an insertion of a targeting moiety amino acid sequence in place of the amino acid sequence from or between positions 213-306 as compared to a polypeptide comprising an amino acid sequence of SEQ ID NO: 1;
an insertion of a targeting moiety amino acid sequence in place of the amino acid sequence from or between positions 305-484 as compared to a polypeptide comprising an amino acid sequence of SEQ ID NO: 1;
an insertion of a targeting moiety amino acid sequence in place of the amino acid sequence from or between positions 213-484 as compared to a polypeptide comprising an amino acid sequence of SEQ ID NO: 1;
an insertion of a targeting moiety amino acid sequence in place of the amino acid sequence from or between positions 213-497 as compared to a polypeptide comprising an amino acid sequence of SEQ ID NO: 1; or
an insertion of a targeting moiety amino acid sequence in place of the amino acid sequence from or between positions 232-497 as compared to a polypeptide comprising an amino acid sequence of SEQ ID NO: 1.

19. The engineered viral particle of any one of claims 1-18, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

20. The engineered viral particle of any one of claims 1-19, wherein the targeting moiety is fused to the Ebola virus glycoprotein via a linker, such as a peptide linker.

21. The engineered viral particle of claim 20, wherein the linker comprises an amino acid sequence of SEQ ID NO: 3 or SEQ ID NO: 4.

22. The engineered viral particle of any one of claims 1-21, wherein the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

23. The engineered viral particle of any one of claims 1-22, wherein the targeting moiety is a CD8 binding moiety.

24. The engineered viral particle of claim 23, wherein the CD8 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

25. The engineered viral particle of any one of claim 23 or 24, wherein the CD8 binding moiety is an anti-CD8 DARPin.

26. The engineered viral particle of claim 25, wherein the anti-CD8 DARPin comprises an amino acid sequence as set forth in SEQ ID NO: 2.

27. The engineered viral particle of any one of claims 1-22, wherein the targeting moiety is a CD7 binding moiety.

28. The engineered viral particle of claim 27, wherein the CD7 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

29. The engineered viral particle of any one of claim 27 or 28, wherein the CD7 binding moiety is an anti-CD7 DARPin.

30. An engineered viral particle comprising:
i. an engineered envelope comprising an Ebola virus glycoprotein fused or linked to a targeting moiety, and a gag-pol protein, wherein the Ebola virus glycoprotein fused or linked to a targeting moiety comprises the amino acid sequence of SEQ ID NO: 5, SEQ ID NO: 6, SEQ ID NO: 7, SEQ ID NO: 8, or SEQ ID NO: 9; and
ii. a nucleic acid molecule encoding a polypeptide of interest.

31. The engineered viral particle of claim 30, wherein the viral particle is a lentivirus.

32. The engineered viral particle of any one of claim 30 or 31, wherein the lentivirus is pseudotyped with the Ebola virus glycoprotein.

33. The engineered viral particle of any one of claims 1-32, wherein the polypeptide of interest is a chimeric antigen receptor (CAR), an antigen, an enzyme, a protein, such as a hemoglobin beta chain.

34. A polypeptide molecule comprising a targeting moiety fused or linked to an Ebola virus glycoprotein in place of the glycan cap, and/or the MLD of the Ebola virus glycoprotein.

35. The polypeptide of claim 34, wherein the targeting moiety is fused or linked to the Ebola virus glycoprotein via a linker, such as a peptide linker.

36. The polypeptide of any one of claim 34 or 35, wherein the linker is a glycine/serine linker.

37. The polypeptide of any one of claims 34-36, wherein the linker comprises an amino acid sequence of SEQ ID NO: 3 or SEQ ID NO: 4.

38. The polypeptide of any one of claims 34-37, wherein the targeting moiety is fused to the Ebola virus glycoprotein at a position from or between positions 213-306, 305-484, 213-484 213-497, and 232-497, as compared to a polypeptide comprising an amino acid sequence of SEQ ID NO: 1.

39. The polypeptide of any one of claims 34-38, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

40. The polypeptide of any one of claims 34-39, wherein the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

41. The polypeptide of any one of claims 34-40, wherein the targeting moiety is a CD8 binding moiety.

42. The polypeptide of claim 41, wherein the CD8 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

43. The polypeptide of any one of claim 37 or 38, wherein the CD8 binding moiety is an anti-CD8 DARPin.

44. The polypeptide of claim 39, wherein the anti-CD8 DARPin comprises an amino acid sequence as set forth in SEQ ID NO: 2.

45. The polypeptide of any one of claims 34-40, wherein the targeting moiety is a CD7 binding moiety.

46. The polypeptide of claim 45, wherein the CD7 binding moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

47. The polypeptide of any one of claim 45 or 46, wherein the CD7 binding moiety is an anti-CD7 DARPin.

48. A polypeptide molecule comprising an EBOV GP, or a variant thereof, fused to a targeting moiety via a linker, such as a peptide linker, and comprising an amino acid sequence of SEQ ID NO: 5, SEQ ID NO: 6, SEQ ID NO: 7, SEQ ID NO: 8, or SEQ ID NO: 9.

49. A method of in vivo delivery of a molecule of interest to a target cell, the method comprising administering an engineered viral particle to a subject in need of delivery, wherein the engineered viral particle comprises:
an engineered envelope comprising a recombinant Ebola virus glycoprotein, or a variant thereof, a gag-pol protein, and an engineered targeting moiety for binding to the target cell;
a nucleic acid molecule encoding the molecule of interest; and
wherein the administration of the engineered viral particle delivers the nucleic acid molecule encoding the molecule of interest to the cell.

50. The method of claim 49, wherein the viral particle is pseudotyped with the Ebola virus glycoprotein.

51. The method of claim 50, wherein the pseudotyped viral particle is a pseudotyped lentivirus.

52. The method of claim 51, wherein the Ebola virus glycoprotein comprises a sequence comprising a deletion, a mutation, insertion, or any combination thereof as compared to a polypeptide comprising an amino acid sequence of SEQ ID NO: 1.

53. The method of claim 52, wherein the deletion comprises an amino acid deletion of a glycan cap amino acid sequence, a MLD amino acid sequence, or any combination thereof.

54. The method of any one of claim 52 or 53, wherein the deletion comprises an amino acid deletion of a glycan cap amino acid sequence.

55. The method of any one of claims 52-54, wherein the deletion comprises an amino acid deletion of a MLD amino acid sequence.

56. The method of any one of claims 52-55, wherein the deletion comprises deletion of a glycan cap sequence, a MLD amino acid sequence, or any combination thereof, from or between positions 213-306, 305-484, 213-484, 213-497, or 232-497, as compared to a polypeptide comprising an amino acid sequence of SEQ ID NO: 1.

57. The method of any one of claims 52-56, wherein the deletion comprises deletion of a glycan cap sequence from or between positions 213-306, or 232-306, as compared to a polypeptide comprising an amino acid sequence of SEQ ID NO: 1.

58. The method of any one of claims 52-57, wherein the deletion comprises deletion of a MLD amino acid sequence from or between positions 305-484, or 305-497, as compared to a polypeptide comprising an amino acid sequence of SEQ ID NO: 1.

59. The method of any one of claims 52-58, wherein the deletion comprises deletion of a glycan cap amino acid sequence, a MLD amino acid sequence, or any combination thereof, from or between positions 213-484, 213-497, or 232-497, as compared to a polypeptide comprising an amino acid sequence of SEQ ID NO: 1.

60. The method of any one of claims 52-59, wherein the deletion comprises an amino acid deletion from or between positions 213-306, 305-484, 213-484, 213-497, or 232-497, as compared to a polypeptide comprising an amino acid sequence of SEQ ID NO: 1.

61. The method of any one of claims 52-60, wherein the deletion comprises an amino acid deletion at any position from or between positions 213-306, 305-484, 213-484, 213-497, or 232-497, as compared to a polypeptide comprising an amino acid sequence of SEQ ID NO: 1.

62. The method of claim 52, wherein the mutation comprises an insertion of a targeting moiety amino acid sequence in place of the glycan cap amino acid sequence, a MLD amino acid sequence, or any combination thereof.

63. The method of any one of claim 52 or 62, wherein the mutation comprises an insertion of a targeting moiety amino acid sequence in place of the glycan cap amino acid sequence, a MLD amino acid sequence, or any combination thereof, from or between positions 213-306, 305-484, 213-484, 213-497, or 232-497, as compared to a polypeptide comprising an amino acid sequence of SEQ ID NO: 1.

64. The method of any one of claim 52 or 63, wherein the mutation comprises:
an insertion of a targeting moiety amino acid sequence in place of the amino acid sequence from or between positions 213-306 as compared to a polypeptide comprising an amino acid sequence of SEQ ID NO: 1;
an insertion of a targeting moiety amino acid sequence in place of the amino acid sequence from or between positions 305-484 as compared to a polypeptide comprising an amino acid sequence of SEQ ID NO: 1;
an insertion of a targeting moiety amino acid sequence in place of the amino acid sequence from or between positions 213-484 as compared to a polypeptide comprising an amino acid sequence of SEQ ID NO: 1;
an insertion of a targeting moiety amino acid sequence in place of the amino acid sequence from or between positions 213-497 as compared to a polypeptide comprising an amino acid sequence of SEQ ID NO: 1; or
an insertion of a targeting moiety amino acid sequence in place of the amino acid sequence from or between positions 232-497 as compared to a polypeptide comprising an amino acid sequence of SEQ ID NO: 1.

65. The method of claim 52, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

66. A method of in vivo delivery of a molecule of interest to a target cell, the method comprising administering an engineered viral particle to a subject in need of delivery, wherein the engineered viral particle comprises:
an engineered envelope comprising an Ebola virus glycoprotein fused or linked to a targeting moiety, and a gag-pol protein, wherein the Ebola virus glycoprotein fused or linked to the targeting moiety comprises the amino acid sequence of SEQ ID NO: 5, SEQ ID NO: 6, SEQ ID NO: 7, SEQ ID NO: 8, or SEQ ID NO: 9; and
a nucleic acid molecule encoding a molecule of interest; and
wherein the administration of the engineered viral particle delivers the nucleic acid molecule encoding the molecule of interest to the cell.

67. The method of claim 66, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

68. A method of delivering a peptide of interest to a target cell, such as an antigen presenting cell (“APC”), the method comprising:
contacting an engineered viral particle of any one of claims 1-29 for binding to a target cell with an APC, thereby delivering the peptide of interest to the APC.

69. The method of claim 68, wherein the target cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

70. A cell comprising the polypeptide of interest encoded for by the engineered viral particle of any one of claims 1-33.

71. The cell of claim 70, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

72. A pharmaceutical composition comprising the cell of claim 70.

73. A composition, such as a pharmaceutical composition, comprising the engineered viral particle of any one of claims 1-33 bound to a cell.

74. The composition of claim 73, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.

75. The composition of any one of claim 73 or 74, wherein the cell is an isolated cell.

76. A pharmaceutical composition comprising the engineered viral particle of any one of claims 1-33.

77. A method of treating a disease in a subject, the method comprising administering to the subject the engineered viral particle of any one of claims 1-33, wherein the engineered viral particle expresses the polypeptide of interest in a cell.

78. The method of claim 77, wherein the disease is as provided herein, such as cancer.

79. The method of claim 77 or 78, wherein the cell is a T cell, a CD4+ T cell, a CD8+ T cell, a CD197+ T cell, a CD62L+ T cell, a CD25+ T cell, a CD152+ T cell, a NK cell, a CD16+ NK cell, a CD56+ NK cell, an alpha-beta T cell, a gamma-delta T cell, a lymphoid progenitor cell, a hematopoietic stem cell (HSC), a CD34+ HSC, a CD117+ HSC, a tumor infiltrating lymphocytes (TIL), an exhausted TIL, a CD279+ TIL, a CD366+ TIL, a CD223+ TIL, a myeloid cell, a monocyte, a macrophage, a central memory T cell, a naïve T cell, an activated T cell, a regulatory T Cell (Treg), or a T-CellCD8+CCR7+.


SEQUENCE LISTING (9 sequences)

SEQ ID NO: 1 - protein, 676 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 2 - protein, 157 aa, synthetic construct
DLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAA

SEQ ID NO: 3 - protein, 18 aa, synthetic construct
SAGGGGSGGGGSGGGGSA

SEQ ID NO: 4 - protein, 19 aa, synthetic construct
TGGGGGSGGGGSGGGGSSA

SEQ ID NO: 5 - protein, 779 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYSAGGGGSGGGGSGGGGSASDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAATGGGGGSGGGGSGGGGSSALSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 6 - protein, 693 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAATGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 7 - protein, 601 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYSAGGGGSGGGGSGGGGSASDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAATGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 8 - protein, 588 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYSAGGGGSGGGGSGGGGSASDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAATGGGGGSGGGGSGGGGSSARRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 9 - protein, 607 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYSAGGGGSGGGGSGGGGSASDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGHTPLHLAAFWGHLEIVEVLLKYGADVNAQDKFGKTPFDLAIDNGNEDIAEVLQKAATGGGGGSGGGGSGGGGSSARRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF